(19) United States
(12) Patent Application Publication
(10) Pub. No.: US 2007/0293560 A1
(43) Pub. Date: Dec. 20, 2007

(54) Compounds and compositions

(76) Inventors: Andrew David Abell, Adelaide (AU); James Morriss Coxon, Christchurch (NZ); Shigeru Miyamoto, Kyoto (JP); Matthew Alan Jones, Christchurch (NZ); Axel Thomas Neffe, Berlin (DE); Steven Geoffrey Aitken, Christchurch (NZ); Blair Gibb Stuart, Christchurch (NZ); Janna Marie Nikkel, Christchurch (NZ); James David Morton, Lincoln (NZ); Roy Bickerstaffe, Christchurch (NZ); Lucinda Jane Goodricke Robertson, Portland, OR (US); Hannah Yunyoung Lee, Lincoln (NZ); Matthew Stewart Muir, Christchurch (NZ)

Correspondence Address: JACOBSON HOLMAN PLLC, 400 SEVENTH STREET N.W., SUITE 600, WASHINGTON, DC, 20004, US

(21) Appl. No.: 11/798,924

(22) Filed: May 17, 2007

(30) Foreign Application Priority Data
May 18, 2006 (NZ) 547303

(51) Int. Cl. A61K 31/401; A61K 31/341; A61P 19/10; A61P 25/02; A61P 27/12; A61P 31/12; A61P 37/00; C07D 207/323; C07D 333/22; C07D 307/36; A61P 37/06; A61P 35/00; A61P 27/16; A61P 25/28; A61P 25/00; A61K 31/381

(52) U.S. Cl. 514/423; 514/448; 514/471; 548/537; 549/487; 549/72

(57) ABSTRACT

The invention provides compounds of Formula I: 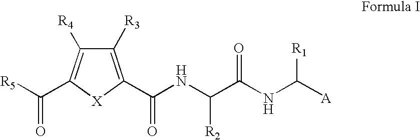 The invention relates to compounds having enzyme inhibitory activity. It further relates to methods for preparing the compounds, compositions comprising them and their use in methods of treatment. Particularly, the invention relates to methods for the treatment or prophylaxis of diseases and disorders resulting from excessive calpain activity.

### TECHNICAL FIELD

This invention relates to compounds with enzyme inhibitory activity and to compositions that comprise one or more of these compounds. In particular, the invention relates to compounds that inhibit calpains. These compounds have utility in the treatment of a variety of diseases.

### BACKGROUND ART

A protease is an enzyme that degrades proteins into smaller peptide fragments. Cysteine proteases incorporate a cysteine residue that is essential to the catalytic process.

Calpains are cysteine proteases that are activated by elevated levels of intracellular calcium ions. Under normal circumstances, calcium ion signalling of calpain leads to controlled proteolysis during cytoskeletal remodelling, signal transduction and apoptosis in mammals. Uncontrolled or high levels of calcium ions in a cell can cause excessive calpain activity, and lead to tissue damage.

There are two major isoforms of calpain, which require different concentrations of calcium ions for activity. They are μ-calpain (also known as calpain I or calpain 1) and m-calpain (also known as calpain II or calpain 2). For example, μ-calpain has been identified as the major isoform present during pathological conditions of the nervous system such as Alzheimer's disease, motor neuron damage, muscular dystrophy and stroke.

The m-calpain isoform has been associated with the development of cataracts. Cataracts are a condition whereby the lens of an eye becomes increasingly clouded and eventually results in blindness. The clouding is due to the precipitation of degraded lens proteins that results from sustained activity of the calcium ion-activated calpain.

Several classes of calpain inhibitors are known. However, many of the known calpain inhibitors have limited therapeutic potential because they have poor stability, cell permeability, solubility or selectivity, or because they have high cell toxicity.

Accordingly, it is an object, of the present invention to go some way to avoiding the above disadvantages or to at least provide the public with a useful choice.

Other objects of the invention may become apparent from the following description which is given by way of example only.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention provides a compound of Formula I or a pharmaceutically acceptable salt, solvate, hydrate or prodrug derivative thereof: 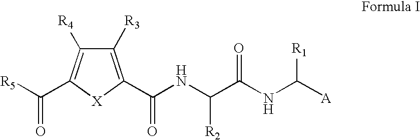
wherein:
X is —NH—, —O— or —S—;
A is —CH₂OH, —CHO or —C(═O)C(═O)NHY;
wherein Y is C₁-C₆ alkyl, hydroxyalkyl or arylalkyl;
   R₁ is a side chain of a natural or non-natural alpha-amino acid;
   R₂ is a side chain of a natural or non-natural alpha-amino acid;
   R₃ and R₄ are independently selected from the group consisting of: —H; -halogen; —NH₂; and —OH; and
   R₅ is H, —OR₆ or —NHR₇;
wherein R₆ is C₁-C₆ alkyl; and
R₇ is C₁-C₆ alkyl.

A particularly preferred group of compounds of Formula I comprises:

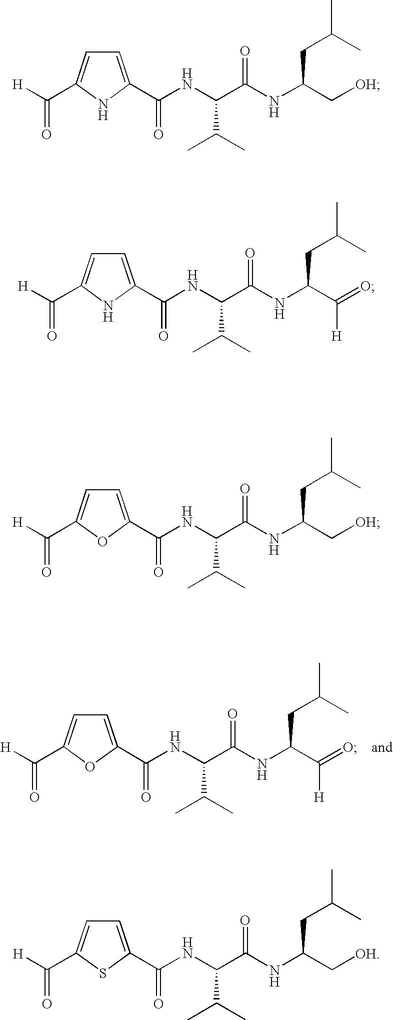

A particularly preferred group of compounds of Formula I comprises:

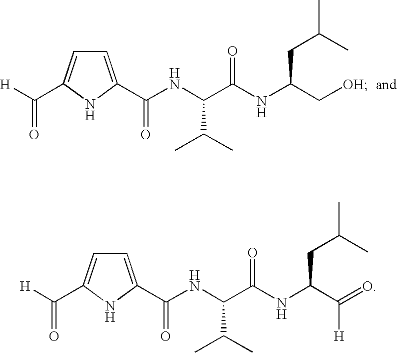

In another aspect, the present invention provides a method for the treatment or prophylaxis of a disease or disorder resulting from excessive calpain activity in a mammal comprising the step of administering a compound of the invention to the mammal.

In another aspect, the present invention provides a pharmaceutical composition comprising a compound of the invention and a pharmaceutically acceptable carrier, diluent or excipient.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The term “comprising”, or variations such as “comprises”, as used in this specification and claims means “consisting at least in part of”. That is to say when interpreting statements in this specification and claims which include that term, the features prefaced by that term in each statement all need to be present but other features can also be present.

Although the present invention is broadly as defined above, those persons skilled in the art will appreciate that the invention is not limited thereto and that the invention also includes embodiments of which the following description gives examples.

### BRIEF DESCRIPTION OF THE DRAWINGS

The invention will now be described with reference to the Figures in which:

FIG. 1 is a graph of the results of a fluorescence-based assay of the inhibition of m-calpain activity by 5-formyl-1H-pyrrole-2-carboxylic acid [(S)-1-((S)-1-hydroxymethyl-3-methyl-butylcarbamoyl)-2-methyl-propyl]-amide 3;

FIG. 2 is a graph of the results of a fluorescence-based assay of the inhibition of m-calpain activity by 5-formyl-1H-pyrrole-2-carboxylic acid [(S)-1-((S)-1-formyl-3-methyl-butylcarbamoyl)-2-methyl-propyl]-amide 6;

FIG. 3 is a photograph of an ovine lens that was pre-incubated with 5-formyl-1H-pyrrole-2-carboxylic acid [(S)-1-((S)-1-formyl-3-methyl-butylcarbamoyl)-2-methyl-propyl]-amide 6 before being treated with calcium chloride; and

FIG. 4 is a photograph of an ovine lens that was treated with calcium chloride.

### DETAILED DESCRIPTION OF THE INVENTION

This invention is directed to compounds with protease inhibitory activity and to compositions that comprise one or more of these compounds.

In a first aspect, the present invention provides a compound of Formula I or a pharmaceutically acceptable salt, solvate, hydrate or prodrug derivative thereof: 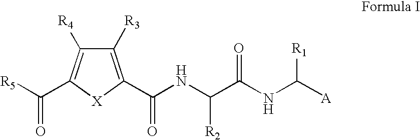
wherein:
X is —NH—, —O— or —S—;
A is —CH₂OH, —CHO or —C(═O)C(═O)NHY;
wherein Y is C₁-C₆ alkyl, hydroxyalkyl or arylalkyl;
   R₁ is a side chain of a natural or non-natural alpha-amino acid;
   R₂ is a side chain of a natural or non-natural alpha-amino acid;
   R₃ and R₄ are independently selected from the group consisting of: —H; -halogen; —NH₂; and —OH; and
   R₅ is H, —OR₆ or —NHR₇;
wherein R₆ is C₁-C₆ alkyl; and
R₇ is C₁-C₆ alkyl.

As used herein, the term “side chain of a natural or non-natural alpha-amino acid” means the group R_{A} in a natural or non-natural amino acid of formula NH₂—CH(R_{A})—COOH.

As used herein, the term “natural alpha-amino acid” includes the 20 L-amino acids (or a residue thereof) which commonly comprise most polypeptides in living systems, that is: alanine (Ala); arginine (Arg); asparagine (Asn); aspartic acid (Asp); cysteine (Cys); glutamine (Gln); glutamic acid (Glu); glycine (Gly); histidine (His); isoleucine (Ileu); leucine (Leu); lysine (Lys); methionine (Met); phenylalanine (Phe); proline (Pro); serine (Ser); threonine (Thr); tryptophan (Trp); tyrosine (Tyr); and valine (Val). The term also includes rarer amino acids found in fibrous proteins (for example, 4-hydroxyproline, 5-hydroxylysine, N-methyllysine, 3-methylhistidine, desmosine and isodesmosine), and naturally occurring amino acids not found in proteins (for example, gamma-aminobutyric acid, homocysteine, homoserine, citrulline, ornithine, canavanine, djenkolic acid and beta-cyanoalanine).

Natural alpha-amino acids which contain functional substituents, for example amino, carboxyl, hydroxy, mercapto, guanidyl, imidazolyl or indolyl groups in their characteristic side chains include arginine, lysine, glutamic acid, aspartic acid, tryptophan, histidine, serine, threonine, tyrosine and cysteine. When R₁ and/or R₂ in the compounds of the invention is a side chain that includes a functional substituent, such as a side chain of one of those natural alpha-amino acids, the functional substituent may optionally be protected. Suitable protecting groups are known to those skilled in the art.

As used herein, the term “unnatural alpha-amino acid” includes any alpha-amino acid (or residue thereof) other than the natural amino acids listed above. Unnatural amino acids include the D-isomers of the natural L-amino acids. Unnatural amino acids also include, but are not limited to: D-phenylalanine; norleucine; hydroxyproline; alpha-carboxyglutamic acid; and pyroglutamic acid.

The prefixes “D-” or “L-” indicate an alpha-amino acid of D- or L-configuration respectively. A “D,L-” prefix indicates a racemic mixture of amino acids of the two configurations. Where no prefix is included, this means that the amino acid can be of either the D- or the L-configuration, except in the Examples where residues are of L-configuration unless otherwise stated.

As used herein, the term “pharmaceutically acceptable salt” is intended to include acid addition salts of any basic moiety that may be present in a compound of Formula I, and base addition salts of any acidic moiety that may be present in a compound of Formula I. Such salts are generally prepared by reacting the compound with a suitable organic or inorganic acid or base. Examples of pharmaceutically acceptable salts of basic moieties include: sulfates; methanesulfonates; acetates; hydrochlorides; hydrobromides; phosphates; toluenesulfonates; citrates; maleates; succinates; tartrates; lactates; and fumarates. Examples of pharmaceutically acceptable salts of acidic moieties include: ammonium salts; alkali metal salts such as sodium salts and potassium salts; and alkaline earth metal salts such as calcium salts and magnesium salts. Other pharmaceutically acceptable salts will be apparent to those skilled in the art.

As used herein, the term “prodrug derivative” is intended to include functional derivatives of the compounds of Formula I, the pharmacological action of which results from conversion to a compound of Formula I by metabolic processes within the body. Therefore, a prodrug derivative is any covalently bonded carrier that releases a compound of Formula I in vivo when the prodrug derivative is administered to a mammal. Prodrug derivatives are generally prepared by modifying functional groups in such a way that the modification is cleaved in vivo to yield the parent compound. Conventional procedures for the selection and preparation of suitable prodrug derivatives are known to those persons skilled in the art and are discussed in, for example, Pro-drugs as Novel Delivery Systems, T. Higuchi and V. Stella in volume 14 of the A.C.S. Symposium Series, 1987, and Bioreversible Carriers in Drug Design, Edward B. Roche (ed.), 1987.

The compounds of Formula I may form hydrates, or solvates with pharmaceutically acceptable solvents. The present invention contemplates such hydrates and solvates as well as the corresponding unsolvated forms.

As used herein, the term “alkyl” is intended to include straight chain, branched chain or cyclic saturated hydrocarbon groups. In a preferred embodiment, the alkyl group is methyl, ethyl, cyclopropyl or n-butyl.

As used herein, the term “aryl” is intended to include aromatic radicals including, but not limited to: phenyl; naphthyl; indanyl; biphenyl; and the like. The term also includes heteroaromatic radicals including, but not limited to: pyrimidinyl; pyridyl; pyrrolyl; furyl; oxazolyl; thiophenyl; and the like.

In a preferred embodiment, X is —NH—.

In a preferred embodiment wherein A is —C(═O)C(═O)NHY, Y is C₁-C₆ alkyl. More preferably, Y is cyclopropyl, ethyl or methyl.

In a preferred embodiment, A is —CH₂OH or —CHO.

In a preferred embodiment, R₁ is a side chain of a natural alpha-amino acid and R₂ is a side chain of a natural alpha-amino acid.

In a preferred embodiment, R₁ is a L-leucine or L-phenylalanine side chain.

In a preferred embodiment, R₂ is a L-valine or L-leucine side chain.

In a particularly preferred embodiment, R₁ is a L-leucine or L-phenylalanine side chain and

R₂ is a L-valine or L-leucine side chain.

In a further particularly preferred embodiment, R₁ is a L-leucine side chain and R₂ is a L-valine sidechain.

In a preferred embodiment, R₃ and R₄ are both —H.

In a preferred embodiment, R₅ is —H.

The compounds of the invention may have asymmetric carbon atoms. Therefore, stereoisomers (both enantiomers and diastereomers) of such compounds can exist. The present invention contemplates the pure stereoisomers and any mixture of the isomers. For example, a pure enantiomer of a compound of the invention can be isolated from a mixture of enantiomers of the compound using conventional optical resolution techniques. Enol forms and tautomers are also contemplated.

A preferred group of compounds of Formula I has the following structural formula:

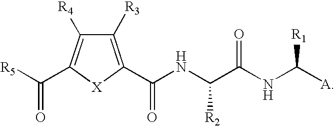

A particularly preferred group of compounds of Formula I has the following structural formula:

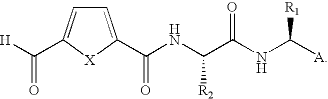

A further particularly preferred group of compounds of Formula I comprises:

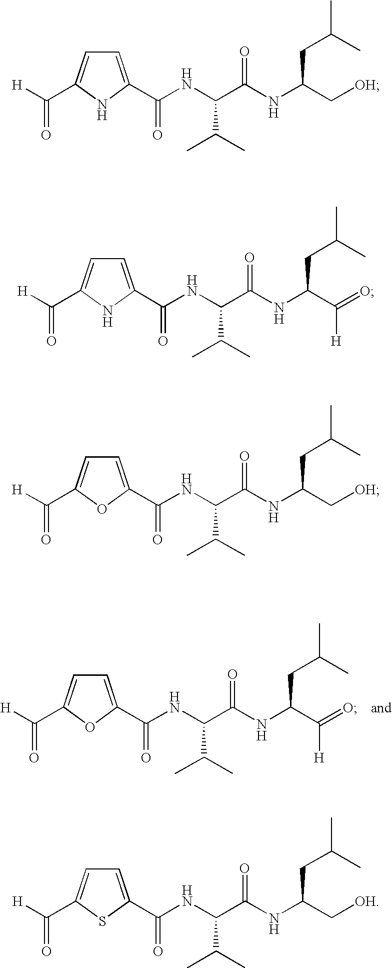

A further particularly preferred group of compounds of Formula I comprises:

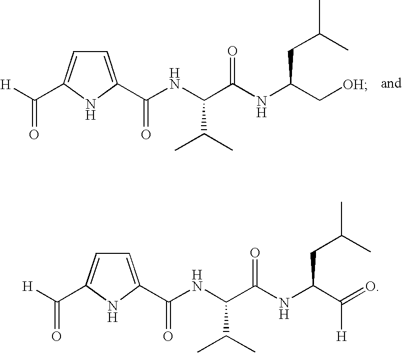

By of example, the compounds of Formula I, wherein A is —CH₂OH or —CHO, may be prepared by a process comprising the steps of:
(a) deprotecting a compound of Formula II: 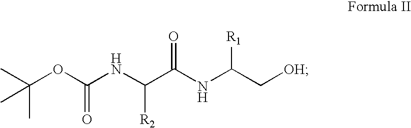
   to provide a compound of Formula III: 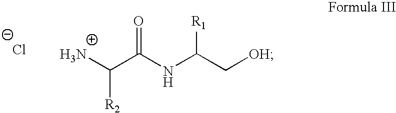
(b) reacting the compound of Formula III with a heterocyclic carboxylic acid of Formula IV: 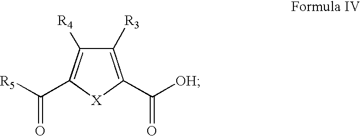
   to provide a compound of Formula V: 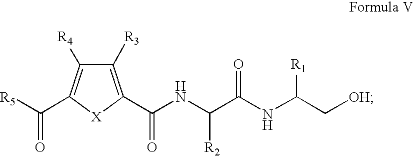
      and, optionally,
(c) oxidising the compound of Formula V to provide a compound of Formula V1: 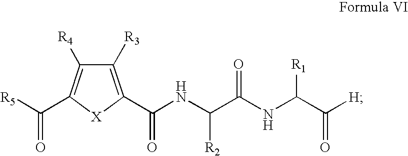
   wherein R₁, R₂, R₃, R₄, R₅ and X are as defined above.

By way of example, the compounds of Formula IV, wherein R₅ is H, may be prepared by the process shown in Scheme 1.

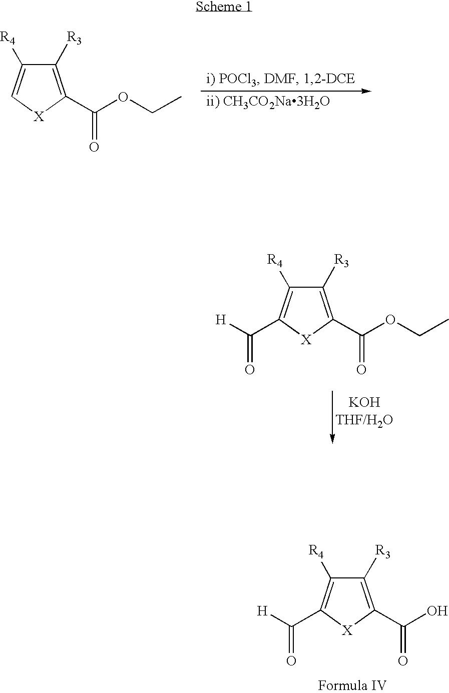

By way of example, the compounds of Formula I, wherein A is —C(═O)C(═O)NHY, may be prepared by a process comprising the steps of:
(a) providing a compound of Formula VII: 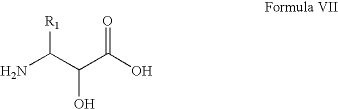
(b) coupling the compound of Formula VII with NH₂—Y to provide a compound of Formula VIII: 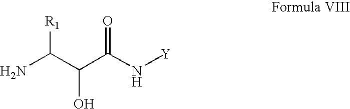
(c) coupling the compound of Formula VIII with a compound of Formula IX: 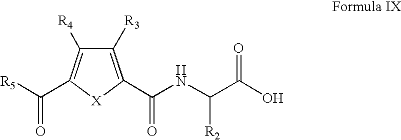
   to provide a compound of Formula X: 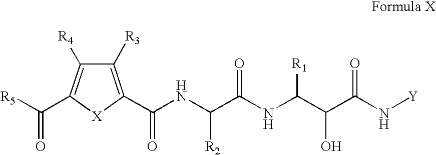
(d) oxidising the compound of Formula X to provide a compound of Formula XI: 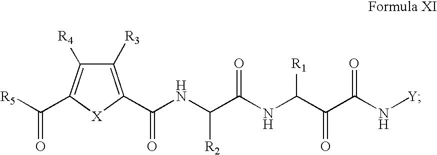
   wherein R₁, R₂, R₃, R₄, R₅, X and Y are as defined above.

By way of example, the compounds of Formula VII may be prepared by the process shown in Scheme 2.

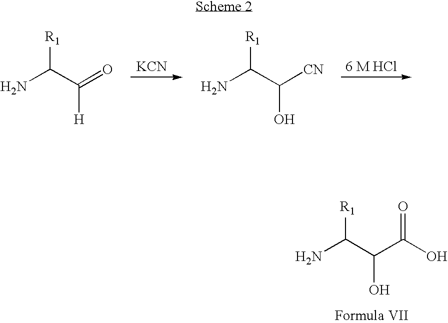

The compound of Formula II may be deprotected using aqueous hydrochloric acid or a solution of hydrogen chloride in an inert organic solvent. Alternative proton sources may also be used, such as trifluoroacetic acid. In one embodiment, the compound of Formula II is deprotected with a solution of hydrogen chloride in diethyl ether.

The coupling reactions, between the compound of Formula III and the heterocyclic carboxylic acid of Formula IV, between the compound of Formula VII and NH₂—Y, and between the compound of Formula VIII and the compound of Formula IX, are generally conducted in dimethylformamide (DMF) in the presence of a coupling agent, such as N-(3-dimethylaminopropyl)-N′-ethylcarbodiimide (EDCl), and a suitable base, such as diisopropyl ethyl amine. A catalytic auxiliary nucleophile, such as 1-hydroxybenzotriazole (HOBt), may also be used. Alternatively, the acid chloride, acid fluoride or mixed acid anhydride of the heterocyclic carboxylic acid may be utilised. Other coupling reagents may also be utilised including, but not limited to: N,N′-dicyclohexylcarbodiimide (DCC); O-(7-azabenzotriazol-1-yl)-N,N,N′,N′-tetramethyluronium hexafluorophosphate (HATU); (7-azabenzotriazol-1-yloxy)tripyrrolidinophosphonium hexafluorophosphate (PyAoP); bromotripyrrolidino-phosphonium hexafluorophosphate (PyBroP); and O-(benzotriazol-1-yl)-N,N,N′,N′-tetramethyluronium tetrafluoroborate (TBTU).

The compounds of Formula V and Formula X may be oxidised with a solution of sulfur trioxide/pyridine complex in dimethylsulfoxide (DMSO). Alternative oxidation procedures that may be used include, but are not limited to: Dess-Martin periodinane; DCC in DMSO; and Swern oxidation. Such procedures are described in J. March, Advanced Organic Chemistry, 4^{th} edition, J. March, 1992.

Compounds of the invention may be prepared according to the general methodology described above and in the Examples. A person skilled in the art will be able, without undue experimentation and with regard to that skill and this disclosure, to select appropriate reagents and conditions to modify these methodologies to produce compounds of the invention.

Those persons skilled in the art will appreciate that other synthetic routes may be used to synthesize the compounds of the invention. In addition, those persons skilled in the art will appreciate that, in the course of preparing the compounds of the invention, the functional groups of intermediate compounds may need to be protected by protecting groups. Functional groups which it may be desirable to protect include, but are not limited to: hydroxyl; amino; and carboxylic acid groups. Protecting groups may be added and removed in accordance with techniques that are well known to those persons skilled in the art. The use of protecting groups is fully described in Protective Groups in Organic Chemistry, J. W. F. McOmie (ed.), 1973 and Protective Groups in Organic Synthesis, 2^{nd} edition, T. W. Greene and P. G. M. Wutz, 1991.

As described in the Examples, compounds within the scope of the invention have been determined to have enzyme inhibitory activity in tests which are predictive of such activity in mammals, including humans. Such properties render the compounds of the invention suitable for use, alone or together with other active agents, in a number of therapeutic applications, including calpain inhibition.

Molecular modelling of compounds within the scope of the invention has demonstrated that they are able to form the beta-strand typical peptide secondary structural motif. Without wishing to be bound by theory, it is believed that a beta-strand structure of a compound is required for molecular recognition by, and inhibition of, calpains.

In particular, compounds within the scope of the invention have been found to inhibit calpains. There is experimental evidence to demonstrate the involvement of excessive calpain activity in a variety of pathologies (K. K. W. Wang, and P-W. Yuen, Trends Pharmacol. Sci. 1994, 15, 412; D. Brömme, Drug News Perspect. 1999, 12, 73). Such pathologies include: inflammatory and immunological diseases, for example rheumatoid arthritis, pancreatitis, multiple sclerosis and inflammations of the gastro-intestinal system including ulcerative or non-ulcerative colitis and Crohn's disease; cardiovascular and cerebrovascular diseases, for example arterial hypertension, septic shock, cardiac or cerebral infarctions of ischemic or hemorrhagic origin, ischemia, and disorders linked to platelet aggregation; disorders of the central or peripheral nervous system, for example neurodegenerative diseases including cerebral or spinal cord trauma, sub-arachnoid haemorrhage, epilepsy, ageing, senile dementia including Alzheimer's disease and Huntington's chorea, Parkinson's disease and peripheral neuropathies; osteoporosis; muscular dystrophies; cachexia; proliferative diseases, for example atherosclerosis or recurrence of stenosis; loss of hearing; cataracts; organ transplant; auto-immune and viral diseases, for example lupus, AIDS, parasitic and viral infections, diabetes and its complications and multiple sclerosis; and cancer.

Given the role of calpains in these pathologies, the compounds within the scope of the invention can produce beneficial or favourable effects in their treatment.

Therefore, the compounds of Formula I may be used in therapy, particularly for inhibiting calpains.

The compounds of Formula I may be used in a method for inhibiting a calpain in a mammal, comprising the step of administering a compound of the invention to the mammal.

The term “mammal” as used herein refers to a human or non-human mammal. Examples of non-human mammals include livestock animals such as sheep, cows, pigs, goats, rabbits and deer; and companion animals such as cats, dogs, rodents and horses.

In another aspect, the present invention provides a method for the treatment or prophylaxis of a disease or disorder resulting from excessive calpain activity in a mammal, comprising the step of administering a compound of the invention to the mammal.

In particular embodiments, the disease or disorder resulting from excessive calpain activity is selected from the group consisting of: disorders of the central or peripheral nervous system; muscular dystrophies; cachexia; loss of hearing; and cataracts.

In a particularly preferred embodiment, the disease or disorder resulting from excessive calpain activity is cataracts.

Therefore, a preferred embodiment of the invention provides a method for the treatment or prophylaxis of cataracts in a mammal, comprising the step of administering a compound of the invention to the mammal.

The compounds of the invention may also be used in an in vitro method for inhibiting a calpain, which comprises contacting the calpain with the compound.

The compounds of the invention may also be used in a method of inhibiting a calpain in a cell, which comprises contacting the cell with an effective amount of the compound.

The invention also provides compositions comprising a compound of the invention. In a preferred embodiment, the composition is a pharmaceutical composition and further comprises a pharmaceutically acceptable carrier, diluent or excipient.

Pharmaceutically acceptable carriers, diluents and excipients are nontoxic to recipients at the dosages and concentrations employed. Each carrier, diluent and exipient must also be “acceptable” in the sense of being compatible with the other ingredients of the formulation.

The compound of the invention, or the composition comprising same, may be administered to a mammal by different routes. The most suitable route may depend upon, for example, the condition and disease of the mammal. Preferred administration routes are oral, parenteral and topical, including intraocular.

The compositions of the present invention may be formulated for administration in unit dosage forms, such as tablets, capsules, pills, powders, granules, suppositories, sterile parenteral solutions or suspensions, sterile non-parenteral solutions or suspensions, oral solutions or suspensions, topical solutions or suspensions, and intraocular solutions or suspensions and the like, that comprise a compound of the invention as an active ingredient.

Solid or fluid unit dosage forms can be prepared for oral administration.

Powders may be prepared by comminuting the active ingredient to a suitably fine size and mixing with a similarly comminuted diluent or excipient. Suitable diluents and excipients are known to those persons skilled in the art.

Capsules may be produced by preparing a powder mixture as described above and filling into formed gelatine sheaths. Soft gelatine capsules may be prepared by encapsulating a slurry of the active ingredient with an acceptable vegetable oil, light liquid petrolatum or other inert oil or triglyceride.

Tablets may be made by preparing a powder mixture, granulating or slugging, adding a lubricant and pressing into tablets. The powder mixture is prepared by mixing the active ingredient, suitably comminuted, with a diluent or base. Suitable diluents and bases are known to those persons skilled in the art. The powder mixture can be granulated by wetting with a binder and forcing through a screen. As an alternative to granulating, the powder mixture can be slugged, i.e. run through a tablet machine and the resulting imperfectly formed tablets broken into pieces (slugs). The slugs can be lubricated to prevent sticking to the tablet-forming dies. The lubricated mixture can then be compressed into tablets.

In one embodiment, the tablet is provided with a protective coating.

Fluid unit dosage forms for oral administration, such as syrups, elixirs and suspensions, wherein a specific volume of composition contains a predetermined amount of active ingredient for administration, can be prepared. Water-soluble active ingredients can be dissolved in an aqueous vehicle together with other ingredients to form a syrup. An elixir is prepared by using a hydro-alcoholic vehicle. Suspensions can be prepared from insoluble forms in a suitable vehicle with the aid of a suspending agent.

Fluid unit dosage forms are prepared for parenteral administration utilising an active ingredient and a sterile vehicle. The active ingredient can be either suspended or dissolved in the vehicle, depending on the form and concentration used. In preparing solutions, the water-soluble active ingredient can be dissolved in a suitable solvent for injection and filter sterilised before filling into a suitable vial or ampoule and sealing. Adjuvants can also be dissolved in the vehicle. Parenteral suspensions are prepared in substantially the same manner.

In addition to oral and parenteral administration, the rectal and vaginal routes may be utilised. An active ingredient can be administered by means of a suppository. A vehicle which has a melting point at about body temperature or one that is readily soluble can be utilised.

Fluid unit dosage forms for intranasal instillation are prepared utilising an active ingredient and a suitable pharmaceutical vehicle. Alternatively, a dry powder can be utilised for insufflation.

The active ingredients, together with a gaseous or liquefied propellant and suitable adjuvants as may be necessary or desirable, can be packaged into a pressurized aerosol container for use as an aerosol.

Suitable dosage forms for intraocular administration include, but are not limited to: eye drops; and ophthalmic emulsions and ointments. In addition to a compound of the invention, the topical dosage forms may comprise a variety of other components, for example: solvents; stabilisers; emulsifiers; suspending agents; surfactants; preservatives; buffers; isotonising agents; pH control agents; and ointment bases.

Examples of the techniques and protocols mentioned above can be found in Remington's Pharmaceutical Sciences, 18^{th} edition, A. R. Gennaro (ed.), 1990.

The compounds and compositions of the invention may be used in combination therapies with one or more other active agents. The one or more other active agents may form part of the same composition, or be formulated as one or more separate compositions for administration at the same time or a different time.

Administration of the compound or composition of the invention is preferably in a therapeutically effective amount, this being an amount sufficient to show the desired benefit to the mammal, including preventing or alleviating the symptoms of any disease or disorder being prevented or treated. The particular dosage of active ingredient to be administered will depend upon the specific disease to be treated, and various characteristics of the mammal, including age, gender, health and weight. In addition, therapeutic factors such as the site of delivery, the method of administration, any concurrent treatment, the frequency of treatment and therapeutic ratio, may also be relevant. Determining the appropriate dosage is within the ability of those persons skilled in the art.

It is expected that a useful unit dosage will comprise between about 0.1 to about 1000 mg, preferably 1 to 200 mg, of a compound of the invention.

When the compound of the invention is formulated for intraocular administration, for example as an eye drop solution, it is expected that a useful concentration of a compound of the invention will comprise about 0.001 to about 2.0% (w/v), preferably 0.01 to 1.0% (w/v). Approximately 20 to 50 μL of such a solution may be instilled into the eye at regular intervals throughout the day.

In a preferred embodiment, the compound of the invention is formulated into an ointment for intraocular administration. In a particularly preferred embodiment, the ointment has the following composition (w/w):

| | |
|---|---|
| 1% | compound of Formula I |
| 25% | cetyl stearyl alcohol |
| 35% | wool fat |
| 39% | paraffinum subl. |

In a preferred embodiment, the compound of the invention is formulated into an emulsion for intraocular administration. In a particularly preferred embodiment, the emulsion has the following composition (w/w):

| | |
|---|---|
| 0.7% | compound of Formula I |
| 20% | cetyl stearyl alcohol |
| 25% | wool fat |
| 25% | paraffinum subl. |
| 1% | sodium lauryl sulfate |
| 0.1% | sodium benzoate |
| 28.3% | water |

The following non-limiting examples are provided to illustrate the present invention and in no way limit the scope thereof.

### EXAMPLES

Compounds within the scope of the invention were prepared by the following synthetic procedures. The synthesis of compounds 3 to 7 is summarised in Scheme 3. 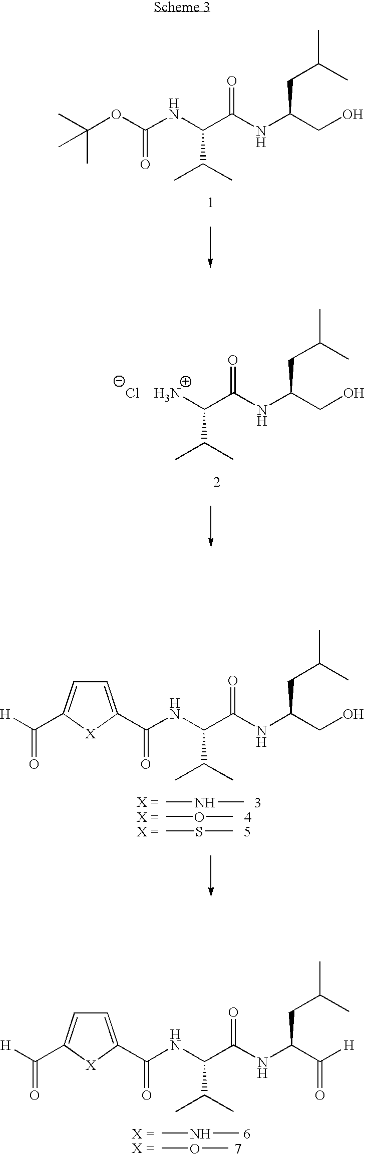
Abbreviations
Boc tert-butoxycarbonyl
BODIPY 4,4-difluoro-5,7-dimethyl-4-bora-3a,4-diaza-s-indacene-3-propionic acid
1,2-DCE 1,2-dichloroethane
DCM dichloromethane
DIPEA diisopropyl ethyl amine
DMF dimethylformamide
DMSO dimethylsulfoxide
EDCl N-(3-dimethylaminopropyl)-N′-ethylcarbodiimide
EMEM Eagle's minimum essential medium
ES electrospray mass spectrometry
Et₂O diethyl ether
EtOAc ethyl acetate
HATU O-(7-azabenzotriazol-1-yl)-N,N,N′,N′-tetramethyluronium hexafluorophosphate
HOBt hydroxybenzotriazole
HRMS high resolution mass spectrometry
LCMS liquid chromatography mass spectrometry
NMR nuclear magnetic resonance spectroscopy
TFA trifluoroacetic acid
THF tetrahydrofuran
   Synthesis

### NH(Boc)-Val-leucinol 1

To a mixture of L-leucinol (2.4 g, 20.5 mmol) and Boc-L-valine (4.4 g, 20.5 mmol) in anhydrous DMF (10 mL) at room temperature, HATU (9.35 g, 24.6 mmol) and DIPEA (8.6 mL, 49 mmol) were added. The reaction mixture was stirred at room temperature overnight. The solution was diluted with EtOAc (100 mL) and was washed with 1M aqueous HCl, water and brine. The organic extract was dried over MgSO₄. Concentration in vacuo afforded a white solid. Purification by re-crystallisation (EtOAc and petroleum ether) gave the dipeptide as a colourless crystalline solid (5.6 g, 87%). rf=0.20 (EtOAc/petroleum ether [1:2]), HRMS (ES+) calcd for C₁₆H₃₃N₂O₄ ([M+H]⁺) 317.2240 found 317.2249; ¹H NMR (500 MHz, (CD₃)₂SO) 0.80-0.94 (12H, m, 4×CH₃), 1.22-1.26 (2H, m, CH₂CH(CH₃)₂), 1.36 (9H, s, (CH₃)₃), 1.58-1.62 (1H, m, CH₂CH(CH₃)₂), 1.85-1.87 (1H, m, CH(CH₃)₂), 3.13-3.17 (1H, m, CH₂OH), 3.28-3.30 (1H, m, CH₂OH), 3.62-3.66 (1H, m, CHCH₂OH), 3.78-3.80 (1H, m, CHCH(CH₃)₂), 6.61 (1H, d, J=8.7 Hz, BocNH), 7.41 (1H, d, J=8.8 Hz, CONh); ¹³C NMR (75 MHz, CDCl₃) 18.0 (CH₃), 19.2 (CH₃), 22.0 (CH₂CH(CH₃)₂), 23.0 (CH₃), 24.7 (CH₃), 28.2 (C(CH₃)₃), 30.4 (CH(CH₃)₂), 39.8 (CH₂CH(CH₃)₂), 49.9 (CHCH₂OH), 60.5 (CHCH(CH₃)₂), 65.5 (CH₂OH), 80.9 (C(CH₃)₃), 156.1 (BocCO), 172.2 (CONH).

### Val-leucinol hydrochloride 2

HCl in Et₂O (2M) (31.6 mL, 63 mmol) was added to NH(Boc)-Val-leucinol (2 g, 6.3 mmol) and the mixture stirred overnight. Concentration in vacuo afforded the hydrochloride salt as a crude white solid that was used without further purification (1.6 g, 99%). mp 64-66° C., ¹H NMR (500 MHz, CD₃OD) 0.92-0.96 (6H, m, 2×CH₃), 1.04-1.08 (6H, m, 2×CH₃), 1.35-1.47 (2H, m, CH₂CH(CH₃)₂), 1.68-1.71 (1H, m, CH₂CH(CH₃)₂), 2.18-2.22 (1H, m, CH(CH₃)₂), 3.45-3.51 (2H, m, CH₂OH), 3.68 (1H, d, J 5.0, CHCH(CH₃)₂), 4.00-4.03 (1H, m, CHCH₂CH(CH₃)₂).

General Procedure for the Coupling of Heterocyclic Carboxylic Acids

To a mixture of Val-leucinol hydrochloride (1 mmol) and a heterocyclic carboxylic acid (1 mmol) in anhydrous DMF (10 mL) at room temperature, EDCl (1.3 mmol), HOBt (1.5 mmol) and DIPEA (4 mmol) were added. The reaction mixture was stirred at room temperature overnight. The solution was diluted with EtOAc (100 mL) and was washed with 1M HCl, water and brine. The organic extract was dried over MgSO₄. Concentration in vacuo afforded a white solid.

### 5-Formyl-1H-pyrrole-2-carboxylic acid [(S)-1-((S)-1-hydroxymethyl-3-methyl-butylcarbamoyl)-2-methyl-propyl]-amide 3

Purification by column chromatography (EtOAc/petroleum ether (1:1), rf=0.1) gave the dipeptide as a yellow crystalline solid (5.33 g, 68%). ¹H NMR (500 MHz, CDCl₃) 0.81-0.86 (6H, d, J=6.5 Hz, 2×CH₃), 1.01-1.06 (6H, d, J=6.5 Hz, 2×CH₃), 1.37 (2H, m, CH₂CH(CH₃)₂), 1.52-1.57 (1H, m, CH₂CH(CH₃)₂), 1.62 (1H, br s, CH₂OH), 2.01-2.05 (1H, m, CH(CH₃)₂), 3.82-3.86 (2H, m, CH₂OH), 4.16-4.18 (1H, m, CHCH₂CH(CH₃)₂), 4.98-5.02 (1H, m, CHCH(CH₃)₂), 6.82 (1H, s, CHCH_{py}), 6.90 (1H, s, CHCH_{py}), 7.72 (1H, d, J=6.5 Hz, CONH), 7.85 (1H, d, J=6.5 Hz, CONH_{py}), 9.62 (1H, s, CHO_{py}), 11.80 (1H, br s, NH_{py}), m/z (ES) 338 ([M+H]⁺), 100.

### 5-Formyl-furan-2-carboxylic acid [(S)-1-((S)-1-hydroxymethyl-3-methyl-butylcarbamoyl)-2-methyl-propyl]-amide 4

Purification by column chromatography (EtOAc/petroleum ether (2:1), rf=0.3) gave the dipeptide as a white foam (0.18 g, 14%). mp 46-48° C.; HRMS (ES+) calcd for C₁₇H₂₆N₂O₅ ([M+H]⁺) 339.1906 found 339.1920; νₘₐₓ (KBr) 1688 (CONH), 1543 (CH₂OH); ¹H NMR (500 MHz, CDCl₃) 0.89 (6H, d, J=6.3 Hz, CH₂CH(CH₃)₂), 1.04 (6H, d, J=6.5 Hz, CH(CH₃)₂), 1.37-1.43 (2H, m, CH₂CH(CH₃)₂), 1.62 (1H, m, CH₂CH(CH₃)₂), 2.17-2.26 (1H, m, CH(CH₃)₂), 3.60 (1H, dd, J=5.5 Hz and J=11.0 Hz, CH₂OH), 3.71 (1H, dd, J=3.4 Hz and J=11.0 Hz, CH₂OH), 4.05-4.10 (1H, m, CHCH₂OH), 4.38-4.41 (1H, m, CHCH(CH₃)₂), 6.21 (1H, d, J=8.0 Hz, NH), 7.26 (1H, d, J=8.0 Hz, NH), 7.29 (2H, m, 2×CHaromatic), 9.75 (1H, s, CHO); ¹³C NMR (75 MHz, CDCl₃); 18.5, 19.2, 22.3, 22.8, 24.8, 31.5, 39.8, 49.8, 49.9, 58.8, 65.1, 115.9, 121.4, 150.6, 152.5, 157.3, 170.8, 170.9, 178.5

### 5-Formyl-thiophene-2-carboxylic acid [(S)-1-((S)-1-hydroxymethyl-3-methyl-butylcarbamoyl)-2-methyl-propyl]-amide 5

Purification by column chromatography (EtOAc/petroleum ether (7:3), rf=0.26) gave the dipeptide as a yellow glass (94 mg, 17%). ¹H NMR (500 MHz, CDCl₃) 0.86 (3H, d, J=6.4 Hz, CH₃), 0.88 (3H, d, J=4.3 Hz, CH₃), 1.03 (3H, d, J=5.9 Hz, CH₃), 1.04 (3H, d, J=6.4 Hz, CH₃), 1.35-1.43 (2H, m, CH₂CH(CH₃)₂), 1.57-1.67 (1H, m, CH₂CH(CH₃)₂), 2.15-2.28 (1H, m, CH(CH₃)₂), 3.57-3.63 (1H, m, CH₂OH), 3.64-3.73 (1H, m, CH₂OH), 4.00-4.12 (1H, m, CHCH₂OH), 4.40-4.48 (1H, m, CHCH(CH₃)₂), 6.58 (1H, mc, NH), 7.29 (1H, mc, NH), 7.65-7.78 (2H, m, 2×CHaromatic), 9.95 (1H, s, CHO)

General Procedure for the Oxidation of Alcohols 3-5

The alcohol (0.5 mmol) was dissolved in DCM (1 mL) and cooled to 0° C. DIPEA (2 mmol) was added and the reaction stirred at 0° C. for 5 minutes. SO₃.pyridine (2 mmol) was dissolved in DMSO (2 mL) and added drop wise to the alcohol solution. The mixture was stirred until the reaction was complete (usually within 2 hours). The solution was diluted with EtOAc (100 mL) and was washed with 1M HCl, water and brine. The organic extract was dried over MgSO₄. Concentration in vacuo afforded a colourless oil.

### 5-Formyl-1H-pyrrole-2-carboxylic acid [(S)-1-((S)-1-formyl-3-methyl-butylcarbamoyl)-2-methyl-propyl]-amide 6

Purification by column chromatography (EtOAc/petroleum ether (1:1), rf=0.1) gave the dipeptide as a yellow crystalline solid (0.1 g, 56%). mp 70-72° C.; ¹H NMR (500 MHz, CDCl₃) 0.79 (3H, d, J=6.5 Hz, CH₃), 0.82 (3H, d, J=6.5 Hz, CH₃), 1.01 (3H, d, J=6.5 Hz, CH₃), 1.06 (3H, d, J=6.5 Hz, CH₃), 1.23-1.26 (1H, m, CH₂CH(CH₃)₂), 1.63-1.653 (2H, m, CH₂CH(CH₃)₂), 2.01-2.03 (1H, m, CH(CH₃)₂), 4.02-4.04 (1H, m, CHCH₂CH(CH₃)₂), 4.80 (1H, mc, CHCH(CH₃)₂), 6.84-6.88 (1H, m, CHCH), 6.93-6.95 (1H, m, CHCCHO), 7.75 (1H, m, NH), 7.82 (1H, m, NH), 9.58 (1H, s, CHO), 9.60 (1H, s, CHO), 11.82 (1H, br s, NH_{pyrroie}); ¹³C NMR (75 MHz, CDCl₃) 18.9, 19.2, 21.63, 22.8, 24.6, 30.8, 37.2, 57.5, 59.1, 111.9, 121.1, 132.2, 134.2, 160.1, 172.5, 180.6, 199.5

### 5-Formyl-furan-2-carboxylic acid [(S)-1-((S)-1-hydroxymethyl-3-methyl-butylcarbamoyl)-2-methyl-propyl]-amide 7

Purification by column chromatography (EtOAc/petroleum ether (2:1), rf=0.4) gave the dipeptide as a white foam (0.1 g, 56%). mp 39-41° C.; νmax (KBr) 1687 (CONH), 1734 (CHO); ¹H NMR (500 MHz, CDCl₃); 0.90 (6H, d, J=5.0 Hz, CH₂CH(CH₃)₂), 1.03 (6H, d, J=9.9 Hz, CH(CH₃)₂), 1.40-1.47 (2H, m, CH₂CH(CH₃)₂), 1.66-1.72 (1H, m, CH₂CH(CH₃)₂), 2.21 (1H, mc, CH(CH₃)₂), 4.54-4.60 (2H, m, CHCH(CH₃)₂ and CHCHO), 6.85 (1H, d, J=7.1 Hz, NH), 7.26 (2H, mc, CHaromatic), 7.35 (1H, d, J=8.8 Hz, NH), 9.59 (1H, s, CHO), 9.73 (1H, s, Fur-CHO); ¹³C NMR (75 MHz, CD₃OD) 18.4, 19.3, 21.8, 22.9, 24.7, 31.4, 37.3, 57.4, 58.4, 116.0, 121.1, 150.5, 152.6, 157.4, 171.2, 178.5, 199.6; m/z (ES) 372.2 ([M+H]⁺), 100.

Enzyme Inhibition

The peptidic alcohol 3 and aldehyde 6 were assayed against m-calpain using a BODIPY-casein substrate in the fluorescence-based assay procedure of V. F. Thompson, S. Saldana, J. Cong and D. E. Goll, Anal. Biochem. 2000, 279, 170. The results of these assays are presented in Table 1.

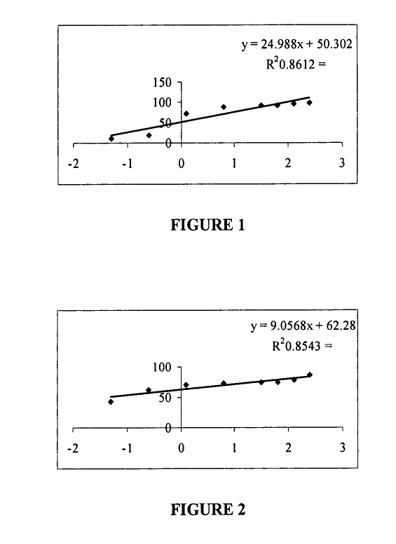

The results of the enzyme inhibition assay for 3 and 6 are graphically represented in FIGS. 1 and 2, respectively.

Cornea Penetration

The peptidic aldehyde 6 was assayed for ovine cornea penetration using a modified Ussing chamber and LCMS based on the procedure of Youn Bok Chung, Kun Han and Vincent H. L. Lee, Pharmaceutical Research, 2000, 15, 1882. Compound 6 (3.5 mg) was dissolved in buffer A (5 mL) and added to the epithelial side a modified Ussing chamber with a pre-mounted adult ovine cornea. Buffer B (5 mL) was added to the endothelial side of the chamber and the whole chamber was incubated 37° C. for 3 hours, 5% CO₂. Samples were taken from both sides of the chamber at time=0 and 180 minutes. The samples were analysed for compound 6 using LCMS. The results of these studies are presented in Table 1.

In Vitro Lens Culture Assay

The ability of the peptidic aldehyde 6 to prevent the formation of a calcium induced cataract in adult ovine lens was assayed using the procedure of J. Sanderson, J. M. Marciantonio and G. A. Duncan, Invest. Opth. Vis. Sci. 2000, 41, 2255.

Three pairs of lenses were tested. One lens from each pair was preincubated with [0.8 μM] 6 for 2 hours while the other was incubated in EMEM-culture media at 35° C., 5% CO₂. Then 5 mM calcium chloride was added onto both the inhibitor treated lens and the other lens, and both lenses were then incubated for 20 hours. The results of these studies are presented in Table 1.

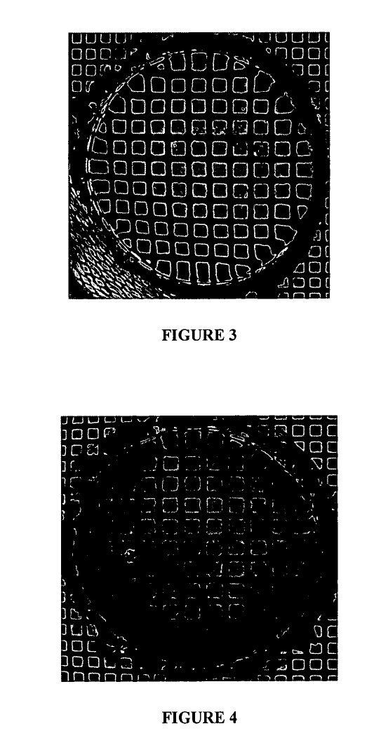

One pair of lenses was photographed when the assay had been completed. FIG. 3 shows the lens that was preincubated with 6 prior to the addition of calcium chloride. FIG. 4 shows the other lens.

TABLE 1

| | | | Cornea | Inhibition of |
|---|---|---|---|---|
| Compound | β-strand^{a} | IC₅₀ (nM) | penetration | cataract formation |
| 3 | yes | 970 | not tested | not tested |
| 4 | yes | 119900 | not tested | not tested |
| 5 | yes | 30400 | not tested | not tested |
| 6 | yes | 30 | yes | yes |
| 7 | yes | not tested | not tested | not tested |

^{a}calculated using Glide, Schrodinger Inc. molecular modelling

In Vivo Tests

An ointment (25 mg) comprising 1% of the peptidic aldehyde 6 was applied twice daily to the left eye of 24 lambs for three months starting when they were two to three months old. The progression of cataracts was determined by a veterinary opthalmologist with a slit-lamp microscope. The treated left eye showed significantly slower cataract progression during the first month following treatment (p<0.05). There were no significant differences between the treated and untreated eyes in the following two months.

An ointment (50 mg) comprising 1% of the peptidic aldehyde 6 was applied to one eye of a lamb, three times in one day. No sign of irritation was observed and the lamb was then sacrificed.

Various body parts were assayed for trace amounts of 6 by LCMS using a method similar to that described by A. Maltese and C. Bucolo, Biomedical Chromatography 2000, 16, 274. A rapid high-performance liquid chromatographic method using a C18 reversed-phase column with UV detection at 254 nm was used for the analysis of 6 in ovine samples. A water solution containing 2% ZnSO₄.7H₂O was used to deproteinise the samples. The mobile phase consisted of CH₃CN and H₂O containing 0.05% TFA. The results are shown in Table 2.

TABLE 2

| | Compound 6 | Amount detected |
|---|---|---|
| Sample | detected | (ng) |
| Aqueous humour (treated eye) | yes | 8 |
| Lens (treated eye) | yes | 6 |
| Vitreous humour (treated eye) | yes | 4 |
| Plasma | no | — |
| Kidney | no | — |
| Liver | no | — |

Formulations
Ointment

An ointment, suitable for intraocular application, and having the following composition (w/w) was prepared:

| | |
|---|---|
| 1% | compound of Formula I |
| 25% | cetyl stearyl alcohol |
| 35% | wool fat |
| 39% | paraffinum subl. |

Emulsion

An emulsion, suitable for intraocular application, and having the following composition (w/w) was prepared according to the procedure described below:

| | |
|---|---|
| 0.7% | compound of Formula I |
| 20% | cetyl stearyl alcohol |
| 25% | wool fat |
| 25% | paraffinum subl. |
| 1% | sodium lauryl sulfate |
| 0.1% | sodium benzoate |
| 28.3% | water |

The hydrophobic phase (cetyl stearyl alcohol, wool fat, paraffinum subl.) and the hydrophilic phase (sodium lauryl sulfate, sodium benzoate, water) were separately heated to 50° C. The compound of Formula I was added to the hydrophobic phase which was stirred until the compound dissolved. The hydrophilic phase was then added to the hydrophobic phase, and the heating source removed. The mixture was stirred until it reached room temperature. The resulting emulsion was then checked for the absence of crystals by differential scanning calorimetry at the melting point of the compound of Formula I.

### INDUSTRIAL APPLICATION

It will be appreciated from the discussion above that this invention provides novel compounds having calpain inhibitory properties. These compounds may be formulated into pharmaceutical compositions for use in any therapeutic application for which their calpain inhibitory activity make them appropriate. Such therapeutic applications include the prevention or treatment of cataracts.

It is not the intention to limit the scope of the invention to the abovementioned examples only. As would be appreciated by a skilled person in the art, many variations are possible without departing from the scope of the invention (as set out in the accompanying claims).

1. A compound of Formula I or a pharmaceutically acceptable salt, solvate, hydrate or prodrug derivative thereof: 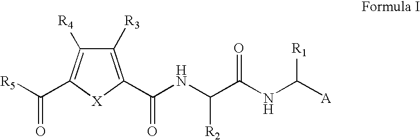
wherein:
X is —NH—, —O— or —S—;
A is —CH₂OH, —CHO or —C(═O)C(═O)NHY;
wherein Y is C₁-C₆ alkyl, hydroxyalkyl or arylalkyl;
R₁ is a side chain of a natural or non-natural alpha-amino acid;
R₂ is a side chain of a natural or non-natural alpha-amino acid;
R₃ and R₄ are independently selected from the group consisting of: —H; -halogen; —NH₂; and —OH; and
R₅ is H, —OR₆ or —NHR₇;
wherein R₆ is C₁-C₆ alkyl; and
R₇ is C₁-C₆ alkyl.

2. The compound as claimed in claim 1, wherein X is —NH—.

3. The compound as claimed in claim 1, wherein R₃ and R₄ are —H.

4. The compound as claimed in claim 1, wherein R₅ is —H.

5. The compound as claimed in claim 1, wherein A is —CH₂OH or —CHO.

6. The compound as claimed in claim 1, wherein A is —C(═O)C(═O)NHY and Y is cyclopropyl, ethyl or methyl.

7. The compound as claimed in claim 1, wherein R₁ is a side chain of a natural alpha-amino acid and R₂ is a side chain of a natural alpha-amino acid.

8. The compound as claimed in claim 1, wherein R₁ is a L-leucine or L-phenylalanine side chain.

9. The compound as claimed in claim 1, wherein R₂ is a L-valine or L-leucine side chain.

10. The compound as claimed in claim 1, wherein R₁ is a L-leucine or L-phenylalanine side chain and R₂ is a L-valine or L-leucine side chain.

11. The compound as claimed in claim 1, wherein R₁ is a L-leucine side chain and R₂ is a L-valine sidechain.

12. A compound as claimed in claim 1, selected from the group consisting of:

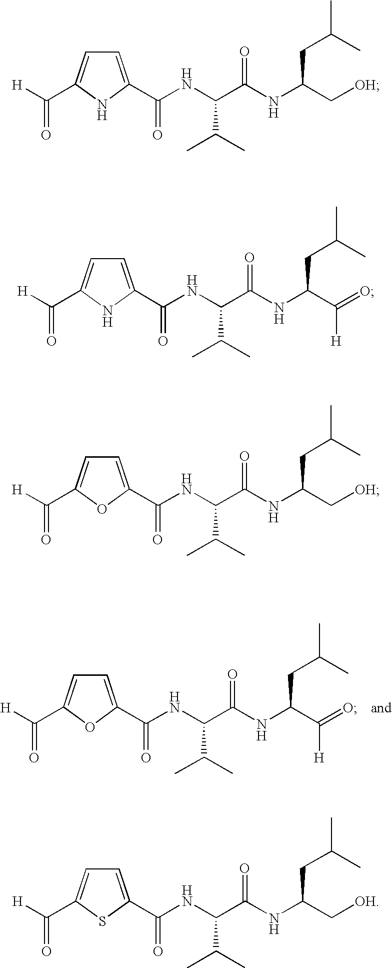

13. A method for the treatment or prophylaxis of a disease or disorder resulting from excessive calpain activity in a mammal, comprising the step of administering a compound as claimed in claim 1 to the mammal.

14. A method as claimed in claim 13, wherein the disease or disorder is selected from the group consisting of: inflammatory and immunological diseases; cardiovascular and cerebrovascular diseases; disorders of the central or peripheral nervous system; osteoporosis; muscular dystrophies; cachexia; proliferative diseases; loss of hearing; cataracts; organ transplants; auto-immune and viral diseases; and cancer.

15. The method as claimed in claim 13, wherein the disease or disorder is selected from the group consisting of: disorders of the central or peripheral nervous system; muscular dystrophies; cachexia; loss of hearing; and cataracts.

16. The method as claimed in claim 13, wherein the disease or disorder is cataracts.

17. A pharmaceutical composition comprising a compound as claimed in claim 1 and a pharmaceutically acceptable carrier, diluent or excipient.

18. The pharmaceutical composition as claimed in claim 17, wherein the composition is formulated for intraocular administration.

19. The pharmaceutical composition as claimed in claim 18, wherein the composition is formulated as an eye drop or an ophthalmic ointment or an emulsion.
